(19) United States
(12) Patent Application Publication
(10) Pub. No.: US 2012/0164271 A1
(43) Pub. Date: Jun. 28, 2012

(54) AUXILLARY ENZYME FOR YEAST, ENZYME DOUGH, BREWING DOUGH AND BREAD, BUN, STEAMED ROLL, BAGEL, METHOD FOR BREWING XIAO BAN ENZYME

(76) Inventor: TAI-SUNG KUO, LINKOU (TW)

(21) Appl. No.: 13/361,964

(22) Filed: Jan. 31, 2012

(30) Foreign Application Priority Data
Sep. 13, 2011 (TW) 100132792

(51) Int. Cl. A21D 8/04; A21D 10/00; A21D 13/00; C12N 9/00

(52) U.S. Cl. 426/18; 435/183; 426/549

(57) ABSTRACT

A fermentation method using an auxiliary enzyme for yeast, enzyme dough, brewing dough and bread, bun, steamed roll, bagel and a method for brewing xiao ban enzyme, and a method for making enzyme yeast are disclosed. The fermentation method includes using xiao ban dough, vegetable and fruit, whole grains and nuts. Xiao ban dough is the fundamental fluid with overall ratio of 3%˜300%. The method for making auxiliary enzyme for yeast includes using vegetable and fruits, whole unlit) and various nuts, and mixing with xiao ban dough. The duration of fermentation process is between 0-90 days, and the fermentation temperature is between 5 Degree Celsius to 30 Degree Celsius. The auxiliary enzyme for yeast can efficiently enhance food color, odor and taste and prolong food preservation.

This application claims the priority benefit of Taiwan patent application serial number TW 100132792 field on Sep. 13, 2011.

### BACKGROUND OF THE INVENTION

1. Field of the Invention

This invention is directed to a method for an auxiliary enzyme for yeast (abbreviated as enzyme yeast), enzyme dough, brewing dough and a method for rapidly fermenting bread, buns, steamed rolls and bagel.

2. Description of Related Art

Some of the current bread does not have artificial butter or emulsifier resulting to a harder and dryer bread. Some bread has emulsifier and natural spices and natural pigment resulting to a softer and good-smelling bread. In some humid and warm climatic countries, in order to prolong the products preservation time, some merchants would add preservatives and antioxidants directly into the dough or into artificial butter. To obtain good-smelling products, spices are used. To obtain luring color, pigments are used. Some merchants add too much food additives which present serious threat to human health. Enzyme is generated from extraction or fermentation. Extraction method is harsh on enzyme, it is dried and without any active ingredients, a product under technology. Fermentation method takes a long time; enzyme is old alter the fermentation process completed; it would also lose most of its active ingredients. In addition, with such a long fermentation process, the production cost is also high.

### SUMMARY OF THE INVENTION

The present invention is directed to a fermentation method using an auxiliary for yeast enzyme dough, brewing dough and bread, steamed bun, a steamed bun with stuffing, bagel.

The present invention is also directed to a method for brewing xiao bran enzyme, and a method for making enzyme yeast.

The fermentation method includes using xiao ban dough, vegetable and fruit, whole grains, nuts, herbs, fresh flowers or fresh meat.

In one embodiment of the present invention, the percentage ration Xiao ban dough is in range between 3%˜300%.

The method for making enzyme yeast includes using vegetable and fruits, whole grain, various nuts, herbs, fresh flowers or fresh meat; and mixing with xiao ban dough.

In one embodiment of the present invention, the duration of fermentation process is between 0-90 days, and the fermentation temperature is between 5 Degree C to 30 Degree C.

The enzyme yeast can efficiently enhance food color, odor and taste and prolong food preservation.

### BRIEF DESCRIPTION OF DRAWINGS

FIG. 1 is a flow chart illustrating preparation of enzyme yeast according to an embodiment of the invention.

FIG. 2 is a flow chart illustrating preparation of xiao ban enzyme according to an embodiment of the invention.

FIG. 3 is a flow chart illustrating a process for making a first type of enzyme dough according to an embodiment of the invention.

FIG. 4 is a flow chart illustrating a process for making a second type of enzyme dough according to an embodiment of the invention.

FIG. 5 is a flow chart illustrating a process for making a third type of enzyme dough according to an embodiment of the invention.

FIG. 6 is a flow chart illustrating a process for making a cooked dough according to an embodiment of the invention.

FIG. 7 is a flow chart illustrating a process for making a raw and edible dough according to an embodiment of the invention.

### DETAIL DESCRIPTION

According to an embodiment of the present invention, kiwi, guava, banana, spinach, onion, chives, orange, citrus, pitaya, pineapple, yam, grapes, pumpkin, sweet potato, bitter gourd, brown rice and millet may be used to produce enzyme yeast. According to another embodiment, marijuana and opium may be used to produce the enzyme yeast. This novel enzyme yeast is not only used to produce superb active enzyme ingredients, it also enhances food deliciousness. In addition, it is easy to prepare this enzyme yeast; it is also less-time consuming and with low production cost.

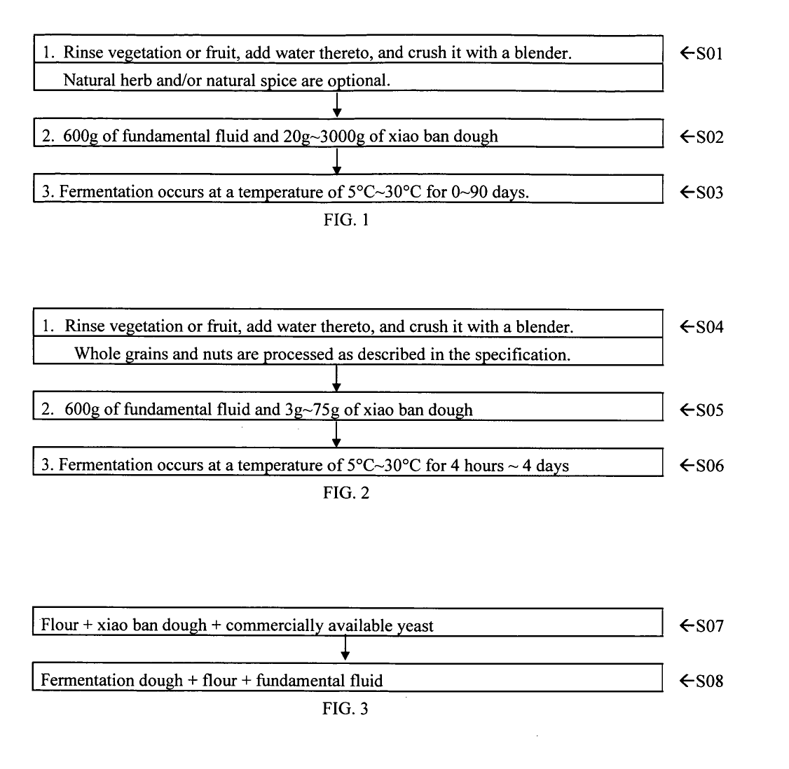

Referring to FIG. 1, in one embodiment, an auxiliary enzyme for yeast may be produced using xiao ban (which is named by the inventor) dough, vegetable and fruit, whole grains and nuts. In one embodiment, the xiao ban dough comprises a weight ratio of 3%˜300% to fundamental fluid. In one embodiment, the fermentation process is from 0-90 days, and the process temperature is between 5 Degree Celsius to 30 Degree Celsius.

In one embodiment, an auxiliary enzyme for yeast may be produced using herbs, fresh flowers or fresh meat.

Referring to FIG. 3 to 5, a dough, such as xiao ban dough, enzyme yeast, base and yeast may be subjected to fermentation, second time dough pressing to produce enzyme dough. The above fermentation process takes about 1-24 hours under the temperature of 5 degree Celsius to 30 degree Celsius. An enzyme is produced from xiao ban dough fermentation base. It is a semiliquid base. Fermentation process may take about 4 hours to 4 days. Fermentation temperature is 3 degree Celsius to 30 degree Celsius. A preservation temperature is under 0 degree Celsius.

In an embodiment, vegetables and fruits may include kiwi, guava, banana, spinach, onion, chives, orange, citrus, pitaya, pineapple, yam, grapes, pumpkin, sweet potato and bitter gourd. In one embodiment, whole grains may include red beans, green beans, soybean and brown rice. In one embodiment, nuts may include walnut, cashew nuts, chestnuts and hazelnuts. When the base is vegetable and fruits, the base may be rinsed clean, added into pure water or mineral water, and grounded in a blender.

When the base is whole grains, the base may soaked in water to germination and then grounded in a blender, or soaked in water to inflate and grounded to pulp or added water and cook to obtain a soft material.

When the base is walnut or cashew nuts, the base is grounded to powder, and water is added, and porridge-like material is obtained.

In one embodiment a perfume, seasoning spice, medicine, detergent or narcotic drug is manufactured from said auxiliary enzyme for yeast.

In one embodiment an enzyme dough includes an enzyme yeast, xiao ban dough, base and flour.

In one embodiment, the base may be selected from vegetables and fruits, whole gains or nuts.

In one embodiment, the vegetable and fruits may include kiwi, guava, banana, spinach, onion, chives, orange, citrus, pitaya, pineapple, yam, grapes, pumpkin, sweet potato and bitter gourd. In one embodiment, the whole grains may include red beans, green beans, soy beans, millets and brown rice.

In one embodiment, nuts may include walnut, cashew nuts, chestnuts or hazelnuts.

In one embodiment, yeast may include any kind sold in the market.

In one embodiment, dough may be prepared by mixing all components and press together, and subjected to fermentation for a predetermined time period.

In one embodiment, fermentation temperature may be in a range of 5 degree Celsius to 30 degree Celsius, and the predetermined fermentation process time may be in a range between 1 to 24 hours.

Referring to FIG. 2, the present invention also provides a type of enzyme generated from a mixture of liquid and semi-liquid fermentation base of xiao ban dough. The xiao ban dough comprises a weight ratio of 5%˜2% to fundamental fluid. The fermentation process takes 4 hours to 4 days. The fermentation temperature is in rage of 5 degree Celsius to 30 degree Celsius. This invention also provides a type of enzyme liquid generated from an enzyme compound base.

In one embodiment, an enzyme may be produced using herbs, fresh flowers or fresh meat.

In one embodiment, natural herbs and natural spices may be added during fermentation. Buckwheat powder or whole wheat powder may also be substituted for flour. Any food products such as bread, steamed roll, bun, or bagel, using this invention's enzyme yeast or dough containing such enzyme yeast is also construed to be within the scope of this invention.

In one embodiment a perfume, seasoning spice, medicine, detergent or narcotic drugs is manufactured from said enzyme.

In one embodiment a perfume, seasoning spice, medicine, detergent or narcotic drug is manufactured from said enzyme liquid.

In one embodiment, brewing dough has longer preservation period, because fermented yeast generates carbon dioxide and alcohol which kill aerobic bacteria. The enzyme content in xiao ban dough and enzyme yeast can speed up the fermentation process and also can form an enzyme compound based from flour and base. In 1 to 12 hours, it can brew eatable raw brewing dough which is in a table form.

The dough, according to the present invention, does not contain any chemical substances. It uses only natural food ingredients which are fermented naturally. Such as kiwi, pineapple, papaya, citrus, guava, banana, spinach, pumpkin or carrot, has various characteristics, hence its fermentation varies. The bread and bun are also unique with different characteristics. This invention aims to brine food hack to its most original and natural balance state.

Below are examples described to better explain this invention. The advantages and merits of this invention will be better clarified with explanations. These examples are for demonstrative purpose only, and do not limit the scope of the present invention. One skilled in the ail would understand that modifications and substitutions can be made to invention's technical details and format when not deviate from the invention's core spirit and parameters; all modifications and substitutions are also construed to be within the scope of the present invention.

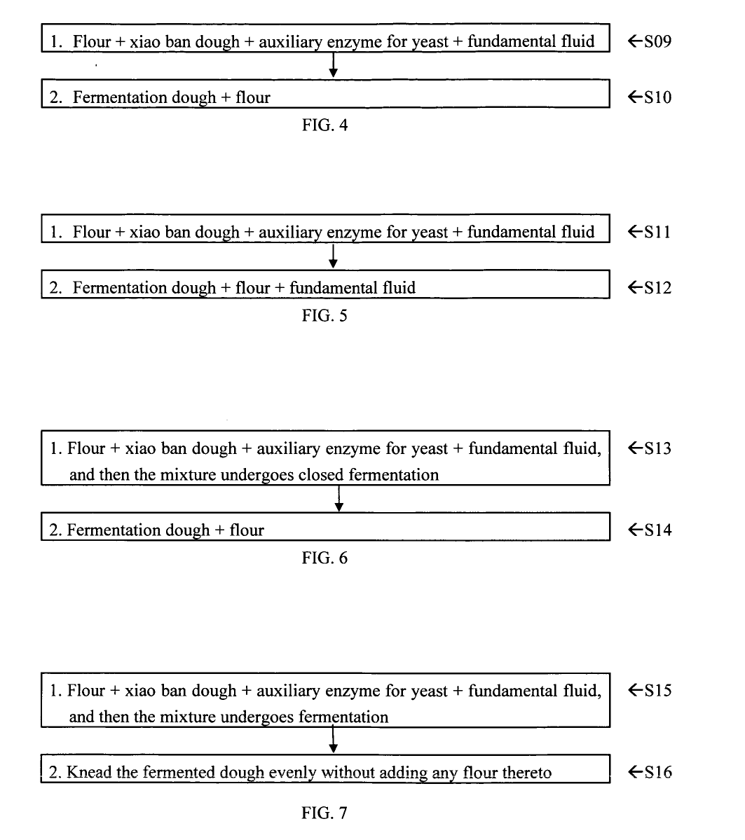

FIG. 1 is a flow chart illustrating preparation of enzyme yeast according to an embodiment of the invention. FIG. 2 is a flow chart illustrating preparation of xiao ban enzyme according to an embodiment of the invention. FIG. 3 is a flow chart illustrating a process for making first type of enzyme dough according to an embodiment of the invention. FIG. 4 is a flow chart illustrating a process for making a second type of enzyme dough according to an embodiment of the invention. FIG. 5 is a flow chart illustrating a process for making a third type of enzyme dough according to an embodiment of the invention. FIG. 6 is a flow chart illustrating a process for making a cooked dough according to an embodiment of the invention. FIG. 7 is a flow chart illustrating a process for making a raw and edible dough according to an embodiment of the invention.

In one embodiment, a base of the enzyme yeast and the xiao ban enzyme may selected from a group comprising guava, citrus, apple, papaya, spinach, carrot, pumpkin or onion. First, rinse the base dealt with tap water to avoid pesticide residue, add in pure water or mineral water in blender for grinding, then add xiao ban dough for fermentation.

When the base is red bean, green bean, soy bean, millet or brown rice, first soak the base in water until germination occurs and then grind the germinated base with a blender. Next, soak the base in water until it inflates. Next, water is added to cook until soil and then cooled off. Next, the resulting material is added in xiao ban dough for fermentation.

When the base is walnut or cashew nut, the base is grounded to powder, and then water is added and stirred until becoming a paste formed, and then the paste is added into xiao ban dough for fermentation.

In one embodiment, for about 3 kg of enzyme yeast:percentage ratio of water, base and xiao ban dough is 0-70%:10-80:10-80% range respectively ratio.

In one embodiment, for about 3 kg of xiao ban enzyme, percentage ratio of water, base and xiao ban dough is 0-80%:20-80:5-12.5% range respectively.

The above enzyme and yeast fermentation temperature is 5 degree c to 30 degree C. Fermentation process takes 0 to 90 days. Preservation temperature is low.

The invention's enzyme dough as described above, can be added with natural herb, natural spices or natural fat.

Bread, steamed bun, steamed bun with stuffing, bagel or doughnut from this invention, can be segmented into separate dough after second pressing. When this segmented dough inflates to 1¼ of original size, they are ready for making. Fillings and fermentation temperature will be determined by dough's active enzyme.

With high active enzyme, those filings easily combine with enzyme or easily cause structural distortion, break chemistry base should be avoided. This will induce flavoring changes immediately.

With high active enzyme, dough finished products should be fermented in low temperature of about 5 degree Celsius to 25 degree Celsius. This is to prolong enzyme activities and dough's inflation force.

In order to prove that the enzyme dough have enzyme activity, the enzyme dough was used for testing break down the starch in Food Industry Research and Development Institute. The starch breakdown enzyme testing were repeated 3 times, result were as follows with 450 nm absorbance value:

| | Dilute 400 | | |
|---|---|---|---|
| Group/OD540 | A | B | C |
| Control Group | 0.0721 | 0.0705 | 0.0696 |
| Sample Group | 0.7132 | 0.7131 | 0.7089 |
| Unit Activity | 295 | 295 | 294 |

In which, by using different concentration standard, standardized curves Y=ax+b were obtained, and unit activity u/g may be expressed as {[(sample group light absorbance value−control group light absorbance value)−b]/ax dilute multiples}/(reaction volume×reaction time)

Combining the above, enzyme breakdown value of unit activity from enzyme dough is 2.9×10²±0.6 u/g falls within the parameter of 1.5×10² to 3.5×10². Therefore, third result would prove that the enzyme dough have starch breakdown enzyme activity and can reduce consumer's body enzyme consumption.

Brewed dough includes cooked brewed dough, raw and edible brewed dough and fermentation liquid as byproduct. Sealed fermentation, instantaneous oxygen depletion, instantaneous kill-off aerobic bacteria and rapid fermentation, this is a benign cycle.

Referring to FIG. 6, method to prepare cooked brewed dough, taking 12 kg of flour for example, first press 6 kg of flour, enzyme yeast content is 0% to 10% of dough weight, commercial yeast content is at 0% to 3.5% of dough weight, xiao ban dough content is 2.5% to 10% of dough weight, fundamental fluid is 0.1% to 10% of dough weieht, water content is 16% to 33% of dough weight, sealed fermentation temperature is 8 degree Celsius to 30 degree Celsius, fermentation process takes 1 to 12 hours.

And add another 6 kg of flour, 0-750 grams of sugar, 5-100 grams of salt, 0-6 pieces of eggs. 0-600 grams of oil are added into fermented dough, and then the dough is pressed until surface appears to soft and shinning. Remaining steps are the same as enzyme dough as described above. No details given here for abbreviation purpose.

Following are 2 kinds of raw and edible enzyme bases:
A: Enzyme Base Compound is a kind of dough in a transitional balanced steady state.
B: The dough that floats on the water surface after a product is produced from Enzyme Base Compound.

Referring to FIG. 7, the preparation method for raw and edible brewed dough A is the same as cooked brewed dough, the only differences are half in water and not adding raw flour during second dough pressing.

The preparation method for raw and eatable brewed dough B is the same as in first dough pressing in cooked brewed dough, prolonging fermentation process until liquid is released, and then floating dough on the water surface is cooped up, and press until the dough surface become soft and shinning.

The above 2 kinds of raw and edible dough, segment it immediately after second pressing, and let segmented dough be fermented to 1¼ size of its original size, start shaping the bread, steamed roll or bagel, package and refrigerate immediately.

While the invention has been described in conjunction with a specific best mode, it is to be understood that many alternatives, modifications, and variations will be apparent to those skilled in the art in light of the foregoing description. Accordingly, it is intended to embrace all such alternatives, modifications, and variations in which fall within the spirit and scope of the included claims. All matters set forth herein or shown in the accompanying drawings are to be interpreted in an illustrative and non-limiting sense.

1. An auxiliary enzyme for yeast, comprising a xiao ban dough, a vegetable or fruit, whole grains, and nuts, allowing a mixture thereof to be produced in a preset duration, wherein a weight percentage of the xiao ban dough in a fundamental fluid ranges between 3% and 300%.

2. The auxiliary enzyme for yeast of claim 1, wherein the mixture is produced in the preset duration of 0-90 days, fermented at a temperature of 5° C.-30° C., and preserved at a temperature below 0° C. continuously.

3. A dough, comprising a xiao ban dough, an auxiliary enzyme for yeast, a fundamental fluid, and a yeast, undergoing second instance kneading for a preset duration, and fermenting until it turns into an enzyme dough.

4. The dough of claim 3, wherein the dough is kneaded for the preset duration of 1-24 hours and fermented at a temperature of 5° C.-30° C.

5. An enzyme for fermenting xiao ban dough for 4 hours to 4 days and at a temperature of 5° C.-30° C. to form a liquid fundamental fluid or a semi-liquid fundamental fluid preserved at a temperature below 0° C.

6. A dough, comprising xiao ban dough, an auxiliary enzyme for yeast, a fundamental fluid, and a yeast, undergoing closed fermentation for 1-12 hours, and undergoing second instance kneading, so as to brew the dough.

7. Dough floating on the water surface after the enzyme base compound of claims 3, 6 has produced a product and squeezed out water therefrom.

8. An enzyme liquid being a liquid squeezed out from a product produced by an enzyme base compound of claims 3, 6.

9. A bread, a steamed bun, a steamed bun with stuffing, and a bagel produced from the dough of claim 1, 3, 6, or 7.

10. A bread, a steamed bun, a steamed bun with stuffing, and a bagel, being produced from the dough of claims 6, 7, and being edible only when cooked or edible raw

11. The dough of claim 1, 3, 5 or 6, wherein fermentation requires a natural herb or a natural spice.
